(19) United States
(12) Patent Application Publication
(10) Pub. No.: US 2018/0125898 A1
(43) Pub. Date: May 10, 2018

(54) MARINE LECITHIN PREPARATIONS WITH ENHANCED OXIDATION RESISTANCE

(71) Applicant: AKER BIOMARINE ANTARCTIC AS, STAMSUND (NO)

(72) Inventors: Gai Ben-Dror, Gita (IL); Ran Numa, Haifa (IL); Shani Sheffer Dee-Noor, Haifa (IL)

(21) Appl. No.: 15/518,031

(22) PCT Filed: Oct. 8, 2015
(86) PCT No.: PCT/IB15/02114

§ 371 Date: Sep. 25, 2017

Related U.S. Application Data
(60) Provisional application No. 62/062,744, filed on Oct. 10, 2014.

(51) Int. Cl. A61K 35/612; A23D 9/013; A23J 7/00; A23L 17/00; A23L 33/12; A23L 33/00; C11B 1/10

(52) U.S. Cl. CPC A61K 35/612; A23D 9/013; A23J 7/00; A23V 2002/00; A23L 33/12; A23L 33/30; C11B 1/10; A23L 17/00

(57) ABSTRACT

The present invention provides marine lecithin preparations including a marine lecithin and one or more exogenous antioxidants. Also provided are a nutritional, pharmaceutical, or nutraceutical composition or a functional or medical food including the marine lecithin preparations. Also provided are processes for making the marine lecithin preparations. Also provided are methods of treating or preventing a disease or disorder, the methods including administering to a subject a therapeutically effective amount of the marine lecithin preparations. Also provided are methods of decreasing an oxidation status of a composition administrated to a subject in need of marine lecithin, the methods including administration of the composition and co-administration of an effective amount of the marine lecithin preparations to the subject in need of marine lecithin.

### FIELD OF THE INVENTION

The present invention relates to marine lecithin preparations with enhanced oxidation resistance.

### BACKGROUND OF THE INVENTION

Epidemiological and clinical studies have associated various health benefits with consumption of fish and sea foods. These positive health outcomes, which are attributed to the presence of long chain n-3 polyunsaturated fatty acids (LC-PUFA), include reduced risk of cardiovascular diseases (CVD) (Hu and Willett 2002), anti-inflammatory properties (Calder 2004), arthritis (Deutsch 2007), premenstrual syndrome, and more.

LC-PUFA are highly susceptible to lipid peroxidation, and can be rapidly oxidized throughout processing as well as following encapsulation or formulation (Hamilton 1998; Undeland et al. 1998; Baik et al. 2004)

Lipid peroxidation refers to the oxidative degradation of lipids. It is a free-radical-mediated chain of reactions that, once initiated, results in an oxidative deterioration of polyunsaturated lipids. These reactions can be initiated or enhanced by a number of toxic products, including hydroperoxides (LOOH) and aldehydes. The progress of the lipid peroxidation process can be monitored by measuring the products of the lipid peroxidation such as LOOH and malondialdehyde (MDA).

Primary peroxidation products are hydroperoxides in which double bond(s) may have moved and/or changed configuration. These products may be structurally rearranged and convert into secondary peroxidation products, resulting in either smaller molecules by fission or larger molecules by dimerization. The lipid hydroperoxides are unstable and their fragmentation yields products such as MDA.

MDA, a three-carbon, low-molecular weight aldehyde, is one of the secondary products of lipid oxidation, is noxious to health, and is formed as a result of the oxidation process of fatty acids containing at least three double bonds. Although MDA is not a stoichiometric marker for lipid peroxidation, its accumulation reflects the intensity of the lipid peroxidation process, as compared to LOOH accumulation. The measurement of these species, along with some others, enables the assessment of the oxidation pathways at different stages, providing detailed information of this dynamic process.

Lipid peroxidation is a key degradation process responsible for the reduction in qualities of marine omega-3 oils, including changes in odor, pigment, and texture. Lipid peroxidation is also responsible for the production of significant amounts of cytotoxic compounds (Addis 1986; Kubow 1992). Furthermore, it has been suggested that oxidized marine omega-3 oils may present an impaired efficacy resulting in variation of supplement effectiveness (Turner 2006). Importantly, several studies have presented increases in plasma peroxidation lipid markers following the consumption of meals or oils containing lipid oxidation end products, indicating that oxidized lipids have the ability to be absorbed by the intestine. Strapans and co-workers (1994, 1999) showed that dietary oxidized lipids are a source of oxidized lipids in chylomicrons of human serum. Naruszewicz found that consumption of heat-treated oil led to a significant increase in postprandial plasma levels of lipid peroxides (Naruszewicz et al. 1987).

Marine lecithins, such as krill oils, contain a natural antioxidant (Astaxanthin) in addition to phospholipid (mostly phosphatidylcholine) bound LC-PUFA. These lecithins are known to be stable throughout their shelf life, probably due to the presence of the natural antioxidant. The inventors of the present application found that marine lecithins which demonstrate stability during their shelf life, with minimal peroxidation, were susceptible to lipid peroxidation in gastrointestinal conditions. The inventors were able to solve this problem by the addition of certain antioxidants to marine lecithin which had preferably low trimethylamine N-oxide (TMAO) levels.

### SUMMARY OF THE INVENTION

The present invention provides marine lecithin preparations comprising a marine lecithin and one or more exogenous antioxidants, wherein the preparation has a concentration of the one or more exogenous antioxidants that is 10 mg/kg or more. At times, the concentration of the one or more exogenous antioxidants is 100 mg/kg or more, at times 500 mg/kg or more, at times 1000 mg/kg or more, at times 1200 mg/kg or more, at times 1400 mg/kg or more, at times 1600 mg/kg or more, at times 2000 mg/kg or more, at times 2500 mg/kg or more, at times 3000 mg/kg or more, and at times 4000 mg/kg or more.

Preferably, the one or more exogenous antioxidants are provided in an effective amount to reduce lipid peroxidation in gastrointestinal conditions. Preferably, the marine lecithin is from a krill or fish source. Preferably, the one or more exogenous antioxidants comprise at least one tocopherol. At times, the one or more exogenous antioxidants comprises a mixture of two or more distinct tocopherols (e.g., alpha tocopherol, beta tocopherol, gamma tocopherol and delta tocopherol).

In certain other non-limiting embodiments of the present invention, the preparation has a concentration of endogenous calcium that is more than 700 mg/kg. In certain other non-limiting embodiments of the present invention, the preparation has a concentration of endogenous magnesium that is more than 500 mg/kg. In certain other non-limiting embodiments of the present invention, the preparation has at least 2% (w/w) phospholipids. In certain other non-limiting embodiments of the present invention, the preparation has at least 3% (w/w) EPA and at least 2% (w/w) DHA.

In certain other non-limiting embodiments of the present invention, the preparation has a concentration of trimethylamine N-oxide (TMAO) that is 25 mgN/100 g or less. At times, the TMAO concentration is 15 mgN/100 g or less, at times 10 mgN/100 g or less, at times 7 mgN/100 g or less, at times 5 mgN/100 g or less, at times 3 mgN/100 g or less, and at times 1 mgN/100 g or less. In certain other non-limiting embodiments of the present invention, the preparation has a concentration of trimethylamine (TMA) that is 5 mgN/100 g or less. In certain embodiments of the present invention, the Krill oil has 5 mgN/100 g or less of TMA after three months, preferably six months, of storage at 40° C. or less. In certain other non-limiting embodiments of the present invention, the preparation has a concentration of sodium that is less than 1200 mg/kg. In certain other non-limiting embodiments of the present invention, the preparation has a concentration of free choline that is less than 450 mg/kg and/or a concentration of betaine that is less than 1000 mg/kg and/or a concentration of total amino acids that is less than 0.3 g/100 g.

In certain other non-limiting embodiments of the present invention, following 180 minutes of incubating the preparation in a gastric model, malondialdehyde (MDA) levels in the preparation do not exceed 6 μmole/g, at times 4 μmole/g, at times 2 μmole/g, and at times 0 μmole/g.

In certain other non-limiting embodiments of the present invention, following 180 minutes of incubating the preparation in a gastric model hydroperoxide (LOOH) levels in the preparation do not exceed 10 μmole/g, at times 6 μmole/g, at times 4 μmole/g, at times 2 μmole/g, and at times 0 μmole/g.

In certain other non-limiting embodiments of the present invention, following 180 minutes of incubating the preparation in a gastric model, levels of phospholipids, triglycerides, fatty acids, astaxanthin, or any other active ingredient are at 80% or more of their initial values prior to incubation. Preferably, the levels are at 85% or more, more preferably 90% or more, even more preferably 97% or more, and most preferably 99% or more of their initial values prior to incubation.

In certain other non-limiting embodiments of the present invention, the preparation further comprises an oil. Preferably, the oil is fish oil, algae oil, vegetable oil, or a combination thereof. More preferably, the oil comprises at least one omega 3 fatty acid, wherein the oil has a concentration of the at least one omega 3 fatty acid that is 25% (w/w) or more.

The present invention also provides a nutritional, pharmaceutical, or nutraceutical composition or a functional or medical food comprising any one of the preparations mentioned above.

The present invention also provides a process of making a marine lecithin preparation according to the invention comprising: mixing the marine biomass with at least one organic solvent; separating the at least one organic solvent from the defatted biomass to obtain a liquid phase containing the at least one organic solvent and the marine lecithin; evaporating the at least one organic solvent from the liquid phase; obtaining the marine lecithin; and adding one or more exogenous antioxidants during any stage of the process.

The present invention also provides a process of making a marine lecithin preparation according to the invention comprising: adding one or more exogenous antioxidants during preparation of a biomass; mixing the marine biomass with at least one organic solvent; separating the at least one organic solvent from the defatted biomass to obtain a liquid phase containing the at least one organic solvent and the marine lecithin; evaporating the at least one organic solvent from the liquid phase and obtaining the marine lecithin.

At times, each one of the described processes may further comprise at least one step of washing with water. Optionally, the extracted marine lecithin is dissolved in an organic solvent mixture when the at least one step of washing the extracted marine lecithin with water is conducted. At times, each one of the processes may further comprise concentrating the marine lecithin and optionally mixing with one or more oils.

At times, the amount of the one or more exogenous antioxidants added results in a final concentration of exogenous antioxidants in the marine lecithin preparation of 10 mg/kg or more, at times 100 mg/kg or more, at times 500 mg/kg or more, at times 1000 mg/kg or more, at times 1200 mg/kg or more, at times 1400 mg/kg or more, at times 1600 mg/kg or more, at times 2000 mg/kg or more, at times 2500 mg/kg or more, at times 3000 mg/kg or more, and at times 4000 mg/kg or more. Preferably, the one or more exogenous antioxidants comprise tocopherol. At times, the one or more exogenous antioxidants comprises a mixture of two or more distinct tocopherols (e.g., alpha tocopherol, beta tocopherol, gamma tocopherol and delta tocopherol).

The present invention also provides methods of treating or preventing a disease or disorder comprising administering to a subject a therapeutically effective amount of the preparation as in any one of the marine lecithin preparations of the invention which is disclosed above. Preferably, the disease or disorder is a cardiovascular disease, cognitive disease, inflammation, inflammatory disease, arthritis, depression, or premenstrual syndrome. In certain non-limiting embodiments the subject is suffering from a cardiovascular disease, cognitive disease, inflammation, inflammatory disease, arthritis, depression, or premenstrual syndrome. In certain other non-limiting embodiments of the present invention, the subject is suffering from a gastrointestinal disease or disorder. Gastrointestinal diseases and disorders include: inflammatory bowel disease such as Crohn's disease or ulcerative colitis, peptic ulcers, gastric ulcers, gastro esophageal reflux disease (GERD), and irritable bowel syndrome (IBS).

The present invention also provides methods of decreasing an oxidation status of a composition administrated to a subject in need of marine lecithin including administration of the composition and co-administration of an effective amount of any one of the marine lecithin preparations disclosed above to the subject in need of marine lecithin. In certain non-limiting embodiments the subject is suffering from a cardiovascular disease, cognitive disease, inflammation, inflammatory disease, arthritis, depression, or premenstrual syndrome. In certain other non-limiting embodiments of the present invention, the subject is suffering from a gastrointestinal disease or disorder. Gastrointestinal diseases and disorders include: inflammatory bowel disease such as Crohn's disease or ulcerative colitis, peptic ulcers, gastric ulcers, gastro esophageal reflux disease (GERD), and irritable bowel syndrome (IBS).

### DETAILED DESCRIPTION OF THE INVENTION

The present invention discloses for the first time marine lecithin preparations containing one or more exogenous antioxidants.

The term “lecithin” refers to a lipid composition containing 1% w/w or more phospholipids.

The term “exogenous antioxidants” refers to at least one natural or synthetic antioxidants or a combination thereof which are added to the marine lecithin, and/or to the raw material of the marine lecithin, and/or during any step of the production of the marine lecithin, and which are present at least in part in the marine lecithin preparation.

As used herein, the terms “marine lecithin preparation” and “preparation”, or any lingual variations thereof are interchangeable.

Natural antioxidants are isolated or originated from natural sources, such as, but not limited to: marine source (e.g., fish, fish parts, krill, squid, shrimp, or other crustaceans), plant source (e.g., soybean, sunflower, rapeseed, canola, corn, olives, rosemary, jasmine, fruits, herbs, origanum, Melissa, grapes, ginseng, cranberry, and tea), Microorganism (either wild found, grown by fermentation, or other) and animal source (e.g., egg, bovine milk, sheep wool, beef meat, lard fat, and others).

According to one embodiment the marine lecithin is extracted from krill (krill oil), fish or fish parts, squid, shrimp or other crustaceans.

According to another embodiment the marine lecithin preparation of the invention further comprises an oil. Optionally, the oil is selected from a group comprising: fish oil, algae oil, vegetable oil and any combination thereof. Optionally, the oil comprises omega 3 fatty acids concentration of 25% (w/w) or more, at times 35% (w/w) or more, and at times 50% (w/w) or more.

In one embodiment of the present invention, the marine lecithin preparation contains exogenous antioxidants levels of 10 mg/kg or above, at times 100 mg/kg or above, at times 500 mg/kg or above, at times 1000 mg/kg or above, at times 1200 mg/kg or above, at times 1400 mg/kg or above, at times 1600 mg/kg or above, at times 2000 mg/kg or above, at times 2500 mg/kg or above, at times 3000 mg/kg or above, and at times 4000 mg/kg or above.

According to another embodiment the one or more exogenous antioxidants are selected from a group comprising: tocopherols, tocotrienols, ascorbyl palmitate, ascorbic acid, rosemary extract, carnosic acid, polyphenols, phenols and any combination thereof. The polyphenol and/or phenols may be of natural origin (e.g., from tea, wine, or olives), of synthetic origin, or mixtures thereof and may include: phenolic acid (gallic acid, ellagic acid, tannic acid, caffeic acid, chlorogenic acid, cinnamic acid, ferulic acid and coumarin), tannins, lignins, flavonoid subclass which includes the flavonols (such as quercetin, galangin, kaempferol, myricetin, fisetin, isorhamnetin, pachypodol, rhamnazin, rutin, hydroxyethylrutosides), flavones (acacetin, apigenin, chrysin, diosmetin, tangeritin, luteolin), isoflavones, flavanones (hesperidin, naringenin, silybin, eriodictyol), anthocyanidins, flavanols (catechins, gallocatechin, epicatechin, Epigallocatechin, epigallocatechin gallate (EGCG), Proanthocyanidins, stilbenes, proanthocyanidins (or leucoanthocyanidins), procyanidins, theaflavins, thearubigins flavonols, 3-hydroxyflavanones (such as dihydroquercetin, dihydrokaempferol), isoflavones (such as genistein, daidzein, glycitein), cyanidin, delphinidin, malvidin, pelargonidin, peonidin, petunidin, resveratrol, phenylpropanoids, anthocyanidins dehydrotheasinensin, theasinensin quinone, epitheaflagallin, hydroxytheaflavin, proepitheaflagallin, and Hydroxytyrosol. Preferably, the one or more exogenous antioxidant is tocopherol without any additional antioxidant. Preferably, the one or more exogenous antioxidants comprises a mixture of two or more distinct tocopherols (e.g., alpha tocopherol and beta tocopherol).

According to another embodiment of the present invention, the TMAO levels in the marine lecithin preparations are 25 mgN/100 g or less, at times 15 mgN/100 g or less, at times 10 mgN/100 g or less, at times 7 mgN/100 g or less, at times 5 mgN/100 g or less, at times 5 mgN/100 g or less, at times 3 mgN/100 g or less, and at times 1 mgN/100 g or less.

The terms “endogenous calcium levels” or “endogenous magnesium levels” refer to calcium or magnesium levels which are extracted from the marine lecithin biomass without the addition of natural or synthetic calcium or magnesium.

In one embodiment of the present invention, the marine lecithin preparation contains high endogenous calcium levels, preferably 700 mg/kg or more, low levels of sodium, preferably 1200 mg/kg or less, and/or low TMA levels, preferably 5 mgN/100 g or less.

According to another embodiment of the present invention, the endogenous calcium level in the marine lecithin preparation is above 700 mg/kg, at times above 1000 mg/kg, at times above 1200 mg/kg, at times above 2000 mg/kg, at times above 3000 mg/kg, and at times above 4000 mg/kg.

According to another embodiment of the present invention, the sodium level in the marine lecithin preparation is below 1200 mg/kg, at times below 1100 mg/kg, at times below 1000 mg/kg, at times below 900 mg/kg, at times below 700 mg/kg, and at times below 500 mg/kg. According to another embodiment of the present invention, the levels of calcium in the marine lecithin preparation are higher than the levels of sodium; at times the ratio of Ca/Na is above 1, at times above 2, at times the ratio is above 3, and at times the ratio is above 4.

In one embodiment of the present invention, the TMA level of the marine lecithin preparation does not increase above 5 mgN/100 g, at times 4 mgN/100 g, at times 3 mgN/100 g, and at times 1 mgN/100 g, after storage for at least 4 months. In another embodiment of the present invention, the TMA level does not increase above 5 mgN/100 g, at times 4 mgN/100 g, at times 3 mgN/100 g and at times 1 mgN/100 g, after storage for at least 5 months, for at least 6 months, for at least 7 months, for at least 8 months, for at least 9 months, for at least 10 months, for at least 11 months, or for at least one year.

In one preferred embodiment of the present invention, the TMA level of the marine lecithin preparation does not increase above 5 mgN/100 g, preferably 4 mgN/100 g, more preferably 3 mgN/100 g and most preferably 1 mgN/100 g, during a period of at least 6 months at ambient temperature (20-30° C.), or during a period of at least 3 months at 40° C. or less.

In one embodiment of the present invention, the marine lecithin preparation contains high endogenous magnesium levels of above 500 mg/kg, at times above 750 mg/kg, at times above 1000 mg/kg, and at times above 2000 mg/kg.

In one embodiment of the present invention, the marine lecithin preparation contains low free choline levels, at times less than 450 mg/kg, at times less than 300 mg/kg, at times less than 200 mg/kg, and at times less than 100 mg/kg.

In one embodiment of the present invention, the marine lecithin preparation contains low betaine levels, at times less than 1000 mg/kg or 750 mg/kg, at times less than 500 mg/kg or 250 mg/kg, at times less than 50 mg/kg, and at times less than 10 mg/kg.

In one embodiment of the present invention, the marine lecithin preparation contains low total amino acids levels, at times less than 0.3 g/100 g, at times less than 0.1 g/100 g, and at times less than 0.05 g/100 g.

According to another embodiment of the present invention, the Krill oil preparation contains low levels of the following amino acids: Alanine, Arginine, Aspartic acid, Cystine, Glutamic acid, Glycine, Histidine, Isoleucine, Leucine, Lysine, Methionine, Ornithine, Phenylalanine, Proline, Serine, Hydroxyproline, Threonine, Tryptophan, Tyrosine, Valine. Preferably, the levels of each of one of said amino acids is less than 0.15 g/100 g or 0.1 g/100 g, preferably less than 0.05 g/100 g, more preferably less than 0.04 g/100 g, even more preferably less than 0.02 g/100 g, and most preferably less than 0.006 g/100 g.

According to another embodiment of the present invention, the marine lecithin preparation comprises at least 2% (w/w) phospholipids, at times above 10% (w/w), at times above 25% (w/w), at times above 35% (w/w) phospholipids, at times above 40% (w/w), at times above 45% (w/w), at times above 50% (w/w), and at times above 60% (w/w).

According to another embodiment of the present invention, the marine lecithin preparation comprises at least 3% (w/w) EPA, at times above 5% (w/w) EPA, at times above 6% (w/w) EPA, at times above 8% (w/w) EPA, at times above 10% (w/w) EPA, and at times above 12% (w/w) EPA. According to another embodiment of the present invention, the preparation comprises at least 2% (w/w) DHA, at times above 3% (w/w) DHA, at times above 5% (w/w) DHA, at times above 7% (w/w) DHA, and at times above 10% (w/w) DHA.

The marine lecithin preparation of the present invention may be in the form of fluid oil, powder, granules, wax, paste, oil or aqueous emulsion, and any other form that will enable its use. In a further aspect of the present invention, the marine lecithin preparation is used in conjunction with or is part of a nutritional, pharmaceutical, or nutraceutical composition or a functional or medical food.

The present invention also provides a nutritional, pharmaceutical, or nutraceutical composition or a functional or medical food comprising any one of the marine lecithin preparations mentioned above.

A medical food as used herein is specially formulated and intended for the dietary management of a disease/disorder that has distinctive nutritional needs that cannot be met by normal diet alone.

A nutritional composition as used herein can be any nutritional composition including, but not limited to: human milk fat substitute, infant formula, adult formula, dairy product, milk powder, drinks, shakes, ice cream, biscuit, soy product, bakery, pastry, bread, cake, sauce, soup, prepared food, frozen food, condiment, confectionary, oil, fat, margarine, spread, filling, cereal, instant product, infant food, toddler food, bar, snack, candy, and chocolate product.

A functional food as used herein can be any functional food, including, but not limited to: dairy product, ice-cream, biscuit, soy product, bakery, pastry, cakes and bread, instant product, sauce, soup, prepared food, frozen food, condiment, confectionary, oils and fat, margarine, spread, filling, cereal, instant product, drinks and shake, infant food, bar, snack, candy, and chocolate product.

A nutraceutical composition as used herein can be any nutraceutical, which can be any substance that may be considered as a food or part of a food and provides medical or health benefits, including the prevention and treatment of diseases or disorders. Such nutraceutical compositions include, but are not limited to: a food additive, a food supplement, a dietary supplement, genetically engineered foods (such as for example vegetables, herbal products, and processed foods such as cereals, soups, and beverages), stimulant functional food, clinical nutrition product, medical food, and pharmafood. Dietary supplements may be delivered in the form of soft gel capsules, tablets, syrups, and other known dietary supplement delivery systems.

The pharmaceutical or nutraceutical compositions may be in any of the many dosage delivery forms commonly used in the art. Pharmaceutical compositions suitable for oral administration may be presented as discrete dosage units (such as pills, tablets, pellets, dragées, capsules, or softgel), as a powder or granule, or as a solution, suspension, syrup, or elixir.

Suitable routes of administration for the compositions of the subject invention are oral, buccal, sublingual, enteral via a feeding tube, topical, transdermal, subcutaneous, or parenteral (including subcutaneous, intramuscular, intravenous, and intradermal) administration. In one embodiment, the compounds are administered orally.

The present invention also provides pharmaceutical compositions wherein the marine lecithin preparation is admixed with (pharmaceutically) acceptable auxiliaries, and optionally other therapeutic agents. The auxiliaries must be “acceptable” in the sense of being compatible with the other ingredients of the composition and not deleterious to the recipients thereof.

In one embodiment of the present invention, a pharmaceutical composition of the present invention further comprises at least one additional pharmaceutically active agent.

The present invention provides a process of preparing the marine lecithin preparations of the invention, comprising extraction process, optional washes, and antioxidant enrichment.

In another one of its aspects the invention provides a marine lecithin preparation of the invention for use in reducing CVD risk factors, and/or treating or preventing CVD, and/or improving a condition in a subject suffering from CVD and/or improving a condition in a subject suffering from cognitive disease or disorder, and/or treating or preventing cognitive disease or disorder, and/or treating or preventing inflammation or inflammatory disease and/or improving a condition in a subject suffering from inflammation or inflammatory disease or disorder and/or treating or preventing depression and/or improving a condition in a subject suffering from depression and/or treating or preventing premenstrual syndrome, and/or improving a condition in a subject suffering from premenstrual syndrome.

In one embodiment the invention provides a preparation of the invention for use in reducing weight, blood pressure or heart rate, and/or for improving serum lipid profile.

The stage of extracting the lecithin is optionally performed by adding one or more organic solvents to the marine biomass to form the lecithin extract. The biomass may be in the form of krill meal, fish meal, in the form of fresh or frozen krill or fish, or in the form of fresh or frozen krill or fish that were processed by cooking and decantation to remove some of the water content. During preparation of the biomass, for example the krill meal or fish meal, antioxidants are optionally added. Optionally, these antioxidants are added before, during and/or after the drying stage in case of krill or fish meal production. Optionally, the liquid phase (containing the lecithin dissolved in the organic solvent) is separated from the defatted biomass by centrifugation, filtration, gravity separation, or other means. Optionally, residual lecithin left with the defatted biomass is extracted from the biomass by repeating of the process described above: addition of one or more organic solvents to the defatted biomass and separation of the liquid by the same optional means (i.e. centrifugation, filtration, gravity separation etc.). In case re-extraction is performed the liquid phases obtained from extraction and re-extractions are united to form final liquid phase.

In case filtration is selected as the separation method, the repeated extraction may be performed by simply washing the defatted biomass left as a “filtration cake” after first liquid phase was removed from it. This re-extraction done by washing will be performed, again, by using one or more organic solvent. The filtrates (i.e., the liquid phases) obtained from extraction and re-extractions are united to form final liquid phase.

The final liquid phase is optionally washed by adding water, optionally also adding an organic solvent, mixing the water and the organic solvent with the final liquid phase. After mixing, separation is performed optionally by gravity or by centrifugation forming two distinct phases: organic phase containing the marine lecithin and a second phase containing most of the water (i.e. water phase). The organic phase can be optionally washed again with water and optionally an organic solvent in the same procedure. The final liquid phase, whether washed or not, is optionally subjected to evaporation stage in order to remove the organic solvent and obtain lecithin. Evaporation may be done under reduced pressure.

The ratio between solvent and marine biomass (solvent volume to biomass weight) during the extraction or re-extraction stages is less than 10:1, preferably less than 5:1, more preferably less than 4:1.

Extraction conditions should be controlled and can, optionally, be maintained between 10-60° C., preferably between 30-40° C., and for 1 minute to 10 hours, preferably for 1-3 hours, and more preferably for 2-2.5 hours. The extraction may be done batchwise, for example in a batch reactor, or optionally by a continuous extraction process. Continuous extraction may be done in co-current or counter current mode in continues extraction systems such as those known in the art. The ratio between solvent to marine biomass in continuous extraction is considered as the ratio between the flow rates of those two streams in the system.

The water washes stage may optionally be conducted continuously as well. Optionally, water and organic phases may be mixed by in-line mixer or by CSTR or by mixers-settlers systems. The mixed water-organic phases may be passed through continues or batch gravity separation tanks or, optionally, be separated by continues centrifugation. In case of continues washing, the ratios between water, organic solvent, and final liquid phase will be considered as the ratio between the flow rates of each of the three streams.

The organic solvent optionally comprises organic solvents which comprise, optionally, a mixture of polar and non-polar solvents. Polar solvent may include one or more of: ethanol, methanol, 2-propanol, and butanol. Non polar solvent may include one or more of: hexane, heptane, and petroleum ether. The ratio between polar and non-polar solvents (volume to volume) is preferably 1:99-99:1, more preferably 5:95-50:50, and most preferably 10:90-20:80. Preferred solvent mixture is a hexane and ethanol mixture.

The volume of the water phase that is used for washing the organic phase (containing lecithin dissolved in organic solvents) during the washes stage is optionally less than 100% of the final liquid phase volume, at times less than 50% of the final liquid phase, at times less than 10% of the final liquid phase volume.

The lecithin obtained following evaporation is optionally subjected to a water washes stage in which the lecithin is preferably re-dissolved in an organic solvent to form an organic phase. Water is added, optionally together with the organic solvents, or optionally after them, to the organic phase, mixed together with it, and separated from it optionally by gravity separation or by centrifugation. The water wash stage can optionally be repeated once or several times. Final marine lecithin preparation will be obtained by removing the solvents from the washed lecithin, optionally by evaporation of the organic solvent, preferably under reduced pressure.

The ratio between lecithin and organic solvent that are forming the organic phase when conducting the water washes is at times (lecithin weight to organic solvent volume) 1:1-1:40, at times 1:2-1:30, at times 1:3-1:10, and at times 1:5-1:8.

The organic solvent optionally comprises organic solvents which comprise, optionally, a mixture of polar and non-polar solvents. Polar solvent may include: ethanol, methanol, butanol and such. Non polar solvent may be from the group of one or more of the following: hexane, heptane and others. The ratio between polar and non-polar solvents (volume to volume) is preferably 1:99-99:1, more preferably 5:95-50:50, more preferably 10:90-20:80. Preferred solvent mixture is a hexane and ethanol mixture.

The volume of the water phase that is used for washing the organic phase (containing lecithin+organic solvent) during the washes stage is less than 100% of the organic phase volume, at times less than 50% of the organic phase volume, at times less than 40% of the organic phase volume, at times less than 30% of the organic phase volume, at times less than 20% of the organic phase volume, and at times less than 10% of the organic phase volume.

According to another embodiment the marine lecithin may be processed in order to increase the phospholipid concentration to 40% (w/w) or more, at times 50% (w/w) or more, and at times 60% (w/w) or more. The phospholipid concentration stage may be done using solvents. The concentrated marine lecithin may optionally be mixed with one or more oils to form a marine lecithin preparation in which the phospholipid concentration is optionally 30% (w/w) or more, at times 40% (w/w) or more, and at times 50% (w/w) or more. The one or more oils used for blending may optionally be selected from: fish oil, alga oil, vegetable oil or any combination thereof. Optionally the one or more oils used for blending may comprise omega 3 fatty acid concentrations of 25% (w/w) or more, at times 35% (w/w) or more and at times 50% (w/w) or more. Optionally, the omega 3 fatty acids are EPA and/or DHA. The one or more oil used for blending may optionally be in triglyceride form, in ethyl ester form, in free fatty acids form, or in any combination thereof. Exogenous antioxidant may be added to the marine lecithin in one or more of the process stages: before it is subjected to the phospholipid concentration stage, during phospholipid concentrations stage, during the stage in which the marine lecithin is mixed with the one or more oils, to the marine lecithin preparation which comprises the one or more oils or any combination thereof.

Trimethylamine (TMA) levels in marine lecithin samples were tested by an external laboratory, Nofima BioLab, Norway. Measurements were conducted in Conway dishes according to a modified version of Conway and Byrne's micro-diffusion method (E. J. Conway et al. 1933; K. J. Öbrink 1955).

TMAO levels in marine lecithin samples were tested by an external laboratory, Nofima BioLab, Norway. Measurements were conducted in Conway dishes according to a modified version of Conway and Byrne's micro-diffusion method (E. J. Conway et al. 1933; K. J. Öbrink 1955).

Hydroperoxide level was tested by FOX analysis. Measurements were conducted at 562 nm, the absorbance of the Ferric-Xylenol Orange Complex. (Gay 1999).

Malondialdehyde (MDA) level was tested by modified TBARS test. The thiobarbituric acid-MDA adduct was quantified by HPLC (Fenaille 2001) or spectrophotometer.

Phospholipids (PL) content in marine lecithin samples was analyzed by ³¹P-NMR by 3rd party lab (Spectral Services) or calculated from HPTLC analysis. HPTLC analysis was performed by dissolving the sample with chloroform and methanol solution (95:5 volume to volume), running the sample on HPTLC silica gel plate using an eluent solution containing water, methanol, acetic acid, acetone, and chloroform, and then staining the plate with a staining solution containing water, sulfuric acid, and anhydrous copper sulfate. EPA and DHA contents were analyzed by gas chromatography (modified AOCS official method Ce 1b-89 or modified AOCS Ce 1i-07).

Elemental analysis to determine mineral and metal content in marine lecithin samples was performed by ICP method by POS Bio-sciences, Canada.

Choline and Betaine in marine lecithin samples were analyzed by Eurofins Analytik GmbH, Germany using LC-MS-MS.

Total amino acids content was analyzed by Eurofins Analytik GmbH, according to reference method ISO 13903:2005; EC 152/2009 (F) and ISO 13904:2005; EC 152/2009 (F).

The accelerated stability test is a standard accelerated storage conditions model for drug substances and products (“Stability Testing of New Drug Substances and Products Q1A(R2)”, ICH Harmonised Tripartite Guideline, February 2003).

The term percent (%) as used herein in connection with amounts and concentrations of compounds to mean weight percent.

### EXAMPLES

### Example 1: Production of Marine Lecithin Preparations According to the Invention

### Example 1A

Extraction of oil from Krill meal was performed by addition of 800 ml solvent to 200 gr Krill meal and shaking at about 40° C. for about 2 hours. Solvents mixture contained hexane and ethanol in a volume ratio of about 90:10 respectively. Filtration of the solvents, including extracted oil, from the meal powder was performed using a Buchner vacuum system. The defatted meal powder, left as the “filtration cake”, was washed with additional 400 ml of the same solvent mixture in order to further extract oil left in the defatted meal. All filtrates were united, and the solvents were evaporated under reduced pressure in rotary evaporator, with a bath at about 50° C. for about 1 hour until less than 10 mbar vacuum was achieved and there was no visible boiling in the oily phase. About 50 g of oil were obtained.

About 30 g out of the 50 g of the obtained oil were dissolved in about 930 ml solvent mixture including hexane, ethanol, and water in the following volume ratio: 87.1% hexane, 9.7% ethanol, 3.2% water. The solution was stirred and the organic and water phases were allowed to separate in a separation funnel. To the organic top phase, mixed natural tocopherols were added in an amount equivalent to 0.14% of the final krill oil weight. Following that, the solvents of the organic (top) phase were evaporated to produce Krill oil under reduced pressure in a rotary evaporator, with a bath at about 50° C. for about 1 hour until less than 10 mbar vacuum was achieved and there was no visible boiling in the oily phase.

The described process resulted in krill oil preparation containing: PL=25.8 g/100 g; EPA=8.2 g/100 g; DHA=4.8 g/100 g; TMAO<1 mgN/100 g.

### Example 1B

Krill oil was extracted using continuous industrial unit in counter current flow. The extraction was performed at about 40° C. with solvents mixture containing hexane and ethanol in a volume ratio of about 90:10 respectively. System parameters were set to ensure a flow of 300 kg/hour krill meal and 1140 L/hour solvent. The solvents containing the dissolved oil were separated continuously by gravity from the defatted meal. The solvents were evaporated under reduced pressure at about 50° C. 400 kg of the received oil was dissolved in about 2748 l solvent mixture including hexane, ethanol and water. The solution was stirred and the organic and water phases were allowed to separate. The solvents of the organic (top) phase were evaporated to produce krill oil under reduced pressure.

The obtained oil was subjected to a second wash with same solvent mixture composed of hexane, ethanol and water.

The described process resulted in krill oil preparation containing: PL=36.4 g/100 g; EPA=11.2 g/100 g; DHA=6.5 g/100 g; TMAO<1 mgN/100 g; free choline=87.1 mg/kg; betaine<2 mg/kg; Ca=1800 mg/kg; Na=400 mg/kg and the following amino acid composition:

| Amino acid | g/100 g |
|---|---|
| Alanine | <0.015 |
| Arginine | <0.042 |
| Aspartic acid | <0.017 |
| Cysteine | <0.006 |
| Glutamic acid | <0.021 |
| Glycine | <0.019 |
| Histidine | <0.02 |
| Hydroxyproline | <0.05 |
| Isoleucine | <0.035 |
| Leucine | <0.015 |
| Lysine | <0.014 |
| Methionine | <0.024 |
| Ornithine | <0.05 |
| Phenylalanine | <0.031 |
| Proline | <0.02 |
| Serine | <0.016 |
| Threonine | <0.006 |
| Tryptophan | <0.01 |
| Tyrosine | <0.023 |
| Valine | <0.016 |

### Example 2: Marine Lecithin Stability

The stability of commercial marine lecithin was evaluated according to the recommended condition by the ICH Guideline “Stability Testing of New Drug Substances and Products Q1A”. For the long term stability, marine lecithin was stored at 25±2° C./60%±5% RH for 12 month, under accelerated conditions marine lecithin was stored at 40±2° C./75%±5% RH.

At each testing point the samples were analysed for Peroxide value (PV) (ACCORDING TO US PHARMACOPEIA <401> FATS AND FIXED OILS; EP PHARMACOPEIA 5.0 2.5.5) and p-Anisidine. (according to modified version of (US pharmacopeia <401> Fats and Fixed Oils; EP pharmacopeia 5.0 2.5.36)

As demonstrated in Tables 1 and 2, the tested marine lecithin did not show any decrease in stability as the PV and p-Anisidine levels remained below pharmacopeia accepted levels. This recognized stability of marine lecithin is usually attributed to the presence of natural antioxidants in the lecithin (for example, astaxanthin in krill oil) and is the reason why regularly no exogenous antioxidants are added to marine lecithins.

TABLE 1
Stability Data for Marine lecithin at 25 ± 2° C./60% ± 5% RH

| Month | Peroxide Value (meO2q/Kg) | p-Anisidine |
|---|---|---|
| 0 | <0.2 | 3.5 |
| 3 | <0.2 | 6.0 |
| 6 | <0.2 | 7.5 |
| 9 | <0.2 | 2.6 |
| 12 | <0.2 | 5.3 |

TABLE 2
Stability Data for Marine lecithin at 40 ± 2° C./75% ± 5% RH

| month | PV (meqO2/Kg) | p-Anisidine |
|---|---|---|
| 0 | <0.2 | 3.5 |
| 1 | <0.2 | 0.8 |
| 2 | <0.2 | 5.4 |
| 3 | <0.2 | 4.3 |
| 6 | <0.2 | 7.3 |

### Example 3: Oxidation Resistance Score of Marine Lecithin Preparation of the Invention in Comparison with Conventional Marine Lecithins in a Gastric Model

Marine lecithin preparations according to the invention and conventional marine lecithins were incubated while shaken in simulated gastric fluid (SGF) which contains 2 mg/ml NaCl, 3.2 mg/ml HCl 37%, and 7 mg/ml Pepsine and with 200 μM Ascorbic acid and 50 μM FeCl₃ as oxidation catalysts, at 37° C. for 180 min. The final concentration of the oils was 10 mg/ml. Peroxidation markers hydroperoxide (LOOH, by Ferric-Xylenol Orange Complex—FOX assay) and malondialdehyde (MDA-TBA₂ HPLC analysis) were analyzed following 180 minutes of incubation in the gastric model.

The results, summarized in Table 3, show that marine lecithins under in vitro stomach model conditions tend to oxidize and lipid peroxidation products form in the mixture (LOOH and MDA).

In order to rank the oils according to their resistance to oxidation in gastric model, a score scale was developed. Each sample was ranked 0 to 4 in increasing oxidation resistance order by the following method: LOOH or MDA levels of 3 microM or below were considered low and each provided the oil with a score of 2. LOOH or MDA levels greater than 3 and lower than 10 microM were considered intermediate and provided a score of 1. LOOH or MDA levels of 10 microM or above were considered high and provided a score of 0. Overall oxidation resistance score was calculated as the sum of LOOH and MDA scores and is demonstrated in Table 3 below.

Interestingly, a significant difference between the oils was obtained. Specifically, the addition of mixed natural tocopherols to high TMAO marine lecithin (>25 mgN TMAO/100 g krill oil) reduced the levels of oxidation parameters and thus increased oxidation resistance score from 0 to 1-2. Similarly, low TMAO levels resulted in oil with higher resistance score (1) in comparison with an oil with high TMAO levels and no added tocopherols (0 resistance score). Most importantly, a synergistic effect was obtained between low TMAO levels and the presence of tocopherols, as demonstrated by the high resistance score (4) of the marine lecithin preparation of the invention. This synergistic effect is unexpected especially in light of publications which pointed out that TMAO presence enhances the antioxidative activity of tocopherol. Ishikawa (1978) demonstrated that tocopherol activity was enhanced in the presence of TMAO and resulted in inhibition of autoxidation of methyl linoleate, whereas a dramatic increase in the peroxide value was observed in the absence of TMAO.

Similarly, Ishikawa and Yuki (1975) demonstrated a synergism between tocopherol and TMAO in inhibiting the oxidation of lard kept in the dark at 60° C.

### Example 4: Omega-3 Levels of Marine Lecithin Preparation of the Invention in Comparison with Conventional Marine Lecithin in a Gastric Model

A marine lecithin preparation according to the invention (krill oil) containing more than 1000 mg/kg exogenous mixed natural tocopherols and less than 5 mgN/100 g TMAO) and a conventional marine lecithin (hill oil) containing no exogenous antioxidants and more than 30 mgN/100 g TMAO) were incubated while shaken in simulated gastric fluid (SGF) which contains 2 mg/ml NaCl, 3.2 mg/ml HCl 37%, and 7 mg/ml Pepsine and with 200 μM Ascorbic acid and 50 μM FeCl₃ as oxidation catalysts, at 37° C. for 180 min. Peroxidation markers hydroperoxide (LOOH, by Ferric-Xylenol Orange Complex—FOX assay) and malondialdehyde (MDA-TBA₂ HPLC analysis) were analyzed following 180 minutes of incubation in the gastric model.

Results indicated that omega-3 fatty acid levels—EPA and DHA, were reduced by about 15% in commercial marine lecithin. However, examination of marine lecithin according to the invention demonstrated less than 5% decrease in EPA and DHA levels following gastric model incubation

### Example 5: Selection of Antioxidants for the Preparation of the Invention

A. Preferred Antioxidants for Fish Oil

As demonstrated in example 2, marine lecithin is stable during its shelf life and thus, usually, no antioxidants are added to the lecithin. As opposed to marine lecithin, fish oil is known to be susceptible to oxidation and thus requires the addition of antioxidants (R. J. Hamilton 1998; P. K. J. P. D. Wanasundara et al.).

It is believed that since different antioxidants function by different mechanism, a combination of different antioxidant will create the best antioxidant activity. This assumption was tested by adding four different antioxidants (mixed natural tocopherols, Ascorbyl palmitate, Rosemary extract and D- [symbol] -tocopherol) and their combinations to fish oil (containing 21% w/w EPA and 52% w/w DHA) and evaluating the oil stability in an accelerated model known as Oil Stability Index (OSI) (based on AOCS Official Method Cd 12b-92).

The Oil Stability Index is defined as the length of time before a rapid acceleration of oxidation accrues under the analysis conditions. The measure of the resistance to oxidation is commonly referred to as the induction period. Thus, higher values predicts longer stability period.

Indeed, as shown in Table 4, mixtures of three antioxidants demonstrated the best antioxidant activity, mixtures of two antioxidants provided moderate antioxidant activity and tocopherols alone provided only minimal activity.

TABLE 4
OSI Results of Different Antioxidant Combinations Added to Fish oil

| | | D-α - | | | Induction |
|---|---|---|---|---|---|
| | | tocopherol | Ascorbyl | Rosemary | time |
| | mixed natural | in | palmitate | extract | at 80° |
| | tocopherols in | oil | in oil | in oil | by OSI |
| sample | oil (mg/kg) | (mg/kg) | (mg/kg) | (mg/kg) | (hrs.) |
| 1 | 1000 | — | 2000 | 3000 | 9.15 |
| 2 | 2000 | — | 2000 | 3000 | 8.31 |
| 3 | 2000 | — | 1000 | 3000 | 7.73 |
| 4 | 2000 | — | 2000 | 1500 | 6.71 |
| 5 | — | 2000 | 2000 | 3000 | 6.21 |
| 6 | 2000 | — | 2000 | — | 4.99 |
| 7 | — | 2000 | 2000 | — | 4.48 |
| 8 | — | 2000 | — | — | 2.82 |
| 9 | 2000 | — | — | — | 2.74 |
| 10 | — | — | — | — | 2.48 |

mixed natural tocopherols = Covi-Ox by Cognis;
12.5% Ascorbyl palmitate = Vitablend ™ 160IP by Vitablend;
Rosemary extract = StabilEnhance OSR by Naturex

B. Oxidation Resistance Score of Commercial Krill Oil Preparations Enriched with Astaxanthin in a Stomach Model

Conventional krill oil, containing high concentration of TMAO, was enriched with three concentrations of Astaxanthin and incubated in simulated gastric fluid (SGF) as described in Example 3. Hydroperoxide as peroxidation marker (LOOH, by Ferric-Xylenol Orange Complex—FOX assay—see method above) was analyzed following 180 minutes.

The results, summarized in Table 5, confirm that commercial Krill oil under in vitro stomach model conditions tends to be oxidized and lipid peroxidation products form in the mixture; e.g., hydroperoxide (LOOH). Interestingly, although the results of example 3 demonstrated that the addition of antioxidants (tocopherols) improved the resistance score, only minimal change in LOOH levels was obtained by Astaxanthin enrichment, even when Astaxanthin concentration was increased more than 10 fold. This result is unexpected especially in light of publications which demonstrated, using other models, that astaxanthin has better antioxidant activity in comparison with tocopherols (Hama et al. 2012; Miki W 1991).

C. Oxidation Resistance Score of Different Marine Lecithin Preparations of the Invention

Marine lecithin with low TMAO levels was mixed with different antioxidants and incubated in simulated gastric fluid (SGF). Peroxidation markers hydroperoxide (LOOH, by Ferric-Xylenol Orange Complex—FOX assay) and malondialdehyde (MDA-TBA₂ HPLC analysis) were analyzed following 180 minutes of incubation.

The results, summarized in Table 6, show that while all antioxidants improved oxidation resistance score (elevation of one or two points) in the stomach model, only antioxidants which contain tocopherols resulted in elevation of three to four points in the oxidation score. Interestingly, the oxidation resistance score was comparable if the tocopherols were the only added antioxidants or if tocopherols were added as part of an antioxidant mixture. This result is unexpected in light of the advantage observed using antioxidant mixtures for increasing fish oil stability.

What is claimed is:

1. A marine lecithin preparation comprising:
a marine lecithin; and
one or more exogenous antioxidants, wherein the preparation has a concentration of the one or more exogenous antioxidants that is 10 mg/kg or more.

2. The preparation of claim 1, wherein the concentration of the one or more exogenous antioxidants is 1000 mg/kg or more.

3. The preparation of claim 1, wherein the one or more exogenous antioxidants are provided in an effective amount to reduce lipid peroxidation in gastrointestinal conditions.

4. The preparation of any one of the preceding claims, wherein the preparation has a concentration of trimethylamine N-oxide (TMAO) that is 25 mgN/100 g or less.

5. The preparation of claim 4, wherein the concentration of TMAO is 5 mgN/100 g or less.

6. The preparation of any one of the preceding claims, further comprising an oil.

7. The preparation of claim 6, wherein the oil is selected from a group comprising: fish oil, algae oil, vegetable oil, and a combination thereof.

8. The preparation of claim 7, wherein the oil comprises at least one omega 3 fatty acid, wherein the oil has a concentration of the at least one omega 3 fatty acid that is 25% (w/w) or more.

9. The preparation of any one of the preceding claims, wherein the one or more exogenous antioxidants comprises at least one tocopherol.

10. The preparation of any one of the preceding claims, wherein the one or more exogenous antioxidants comprises a mixture of two or more distinct tocopherols.

11. The preparation of any one of the preceding claims, wherein the marine lecithin is from a krill source or a fish source.

12. The preparation of claim 11, wherein the marine lecithin is from a krill source.

13. The preparation of any one of the preceding claims, wherein the preparation has a concentration of endogenous calcium that is more than 700 mg/kg.

14. The preparation of any one of the preceding claims, wherein the preparation has a concentration of sodium that is less than 1200 mg/kg.

15. The preparation of any one of the preceding claims, wherein the preparation has a concentration of trimethylamine (TMA) that is 5 mgN/100 g or less.

16. The preparation of any one of the preceding claims, wherein following 180 minutes of incubation in a gastric model malondialdehyde (MDA) levels in the preparation do not exceed 6 μmole/g.

17. The preparation of any one of the preceding claims, wherein following 180 minutes of incubation in a gastric model hydroperoxide (LOOH) levels in the preparation do not exceed 10 μmole/g.

18. The preparation of any one of the preceding claims, wherein following 180 minutes of incubation in a gastric model the levels of phospholipids, triglycerides, fatty acids, astaxanthin, or any other active ingredient are at 80% or more of their initial values prior to incubation.

19. A nutritional composition comprising the preparation as in any one of claims 1-18.

20. A nutraceutical composition comprising the preparation as in any one of claims 1-18.

21. A pharmaceutical composition comprising the preparation as in any one of claims 1-18.

22. A dietary supplement composition comprising the preparation as in any one of claims 1-18.

23. A medical food composition comprising the preparation as in any one of claims 1-18.

24. A process of making a marine lecithin preparation comprising a marine lecithin and exogenous antioxidants comprising:
mixing the marine biomass with at least one organic solvent;
separating the at least one organic solvent from the defatted biomass to obtain a liquid phase containing the at least one organic solvent and the marine lecithin;
evaporating the at least one organic solvent from the liquid phase;
obtaining the marine lecithin; and
adding one or more exogenous antioxidants during any stage of the process.

25. A process of making a marine lecithin preparation comprising a marine lecithin and exogenous antioxidants comprising:
adding one or more exogenous antioxidants during preparation of a biomass;
mixing the marine biomass with at least one organic solvent;
separating the at least one organic solvent from the defatted biomass to obtain a liquid phase containing the at least one organic solvent and the marine lecithin;
evaporating the at least one organic solvent from the liquid phase and obtaining the marine lecithin.

26. The process of claims 24 or 25, further comprising at least one step of washing with water.

27. The process of claim 26, wherein the extracted marine lecithin is dissolved in an organic solvent mixture when the at least one step of washing the extracted marine lecithin with water is conducted.

28. The process of any one of claims 24 to 27, further comprising concentrating the marine lecithin

29. The process of claim 28 further comprising, mixing the marine lecithin with one or more oils.

30. The process of any one of claims 24 to 29, wherein the one or more exogenous antioxidants are added to a concentration of 10 mg/kg or more of the marine lecithin preparation.

31. The process of claim 30, wherein the one or more exogenous antioxidants are added to a concentration of 1000 mg/kg or more of the marine lecithin preparation.

32. The process of any one of claims 24 to 31, wherein the one or more exogenous antioxidants comprises tocopherol.

33. The process of any one of claims 24 to 31, wherein the one or more exogenous antioxidants comprises a mixture of two or more distinct tocopherols.

34. A method of treating or preventing cardiovascular diseases, cognitive disease, inflammation, arthritis, depression or premenstrual syndrome, comprising:
administering to a subject a therapeutically effective amount of the preparation as in any one of claims 1 to 23.

35. A method of decreasing an oxidation status of a composition administrated to a subject in need of marine lecithin comprising:
administration of the composition; and
co-administration of an effective amount of a marine lecithin preparation as in any one of claims 1-23 to the subject in need of marine lecithin.

36. The method of claims 34 or 35, wherein the subject is suffering from a gastrointestinal disease or disorder.

37. The method of claim 36, wherein the gastrointestinal disease or disorder is inflammatory bowel disease.

38. The method of claim 37, wherein the inflammatory bowel disease is Crohn's disease or ulcerative colitis.

39. The method of claim 36, wherein the gastrointestinal disease or disorder is peptic and gastric ulcers, gastro esophageal reflux disease (GERD), or irritable bowel syndrome (IBS).
